(19) United States
(12) Patent Application Publication
(10) Pub. No.: US 2012/0134921 A1
(43) Pub. Date: May 31, 2012

(54) SOLID COMPOSITIONS COMPRISING 5-AMINOLEVULINIC ACID

(75) Inventors: Oddveig Sellaeg Helland, Oslo (NO); Gry Stensrud, Nittedal (NO); Bjorn Klem, Oslo (NO); Jon Erik Braenden, oslo (NO); Aslak Godal, Oslo (NO); Jo Klavenes, Oslo (NO)

(73) Assignee: Photocure ASA, Oslo (NO)

(21) Appl. No.: 13/377,692

(22) PCT Filed: Jun. 11, 2010
(86) PCT No.: PCT/EP2010/003531

§ 371 Date: Jan. 12, 2012

(30) Foreign Application Priority Data
Jun. 11, 2009 (EP) 09251538.6

(51) Int. Cl. A61K 51/04; A61K 49/00

(52) U.S. Cl. 424/1.61; 424/9.6

(57) ABSTRACT

This invention relates to solid compositions and solid pharmaceutical products for use in methods of photodynamic diagnosis of cancer, pre-cancerous and non-cancerous conditions in the lower part of the gastrointestinal system. The solid pharmaceutical compositions and pharmaceutical products comprise an active ingredient which is 5-aminolevulinic acid (5-ALA) or a precursor or derivative of 5-ALA or pharmaceutically acceptable salts thereof. The invention relates further to methods of photodynamic diagnosis of cancer, pre-cancerous and non-cancerous conditions of the lower gastrointestinal tract, wherein the solid pharmaceutical compositions and pharmaceutical products are used.

This invention relates to solid compositions and solid pharmaceutical products for use in methods of photodynamic diagnosis of cancer, pre-cancerous and non-cancerous conditions in the lower part of the gastrointestinal system. The solid pharmaceutical compositions and pharmaceutical products comprise an active ingredient which is 5-aminolevulinic acid (5-ALA) or a precursor or derivative of 5-ALA or pharmaceutically acceptable salts thereof. The invention relates further to methods of photodynamic diagnosis of cancer, pre-cancerous and non-cancerous conditions of the lower gastrointestinal tract, wherein the solid pharmaceutical compositions and pharmaceutical products are used.

Photodynamic diagnosis (PDD) is a relatively new technique for the diagnosis of pre-cancerous lesions, cancer and non-cancerous diseases. PDD involves the administration of a photosensitiser or a precursor thereof to an area of interest. The photosensitiser or precursor thereof is taken up into the cells, where a precursor of a photosensitiser is converted into a photosensitiser. Upon exposure of the area of interest to light, the photosensitiser is excited and displays in response a fluorescence which is detected. The photosensitiser accumulates preferentially in metabolically active tissue, such as inflamed or neoplastic tissue; hence such tissue can be distinguished from healthy tissue. The mechanisms are still not fully understood, but studies suggest that accumulation is not due to selective uptake by cancerous cells. Rather, there are similar levels of uptake in all cell types, but the processes of conversion and elimination are different in metabolically active cells, such as neoplastic tissue, leading to a concentration gradient between e.g. inflamed/neoplastic and normal tissue.

Several photosensitisers and precursors of photosensitisers are known and described in the literature. 5-aminolevulinic acid (5-ALA) and certain derivatives thereof, e.g. 5-ALA esters, are precursors of photosensitisers and once taken up into cells, are converted to protoporphyrins, such as protoporphyrin IX (PpIX). Currently, one pharmaceutical product, Hexvix® developed by Photocure ASA (Oslo, Norway), which comprises 5-ALA hexyl ester, is in clinical use for PDD of bladder cancer and pre-cancerous lesions. In the PDD procedure, Hexvix is installed into the bladder in form of an aqueous solution which is freshly prepared on site from a lyophilized power of the 5-ALA hexyl ester and a dissolution medium. This is due to the limited stability of 5-ALA and 5-ALA esters in aqueous media which limits the shelf life of water-containing pharmaceutical products in which they are present.

A number of different strategies have been adopted to try to overcome this problem. For instance, Metvix® by Galderma S. A, an oil-in-water emulsion (cream) for the photodynamic treatment of actinic keratosis and basal cell carcinoma, is stored in cold conditions. Levulan Kerastick® developed by DUSA Pharmaceuticals, a product for the photodynamic treatment of skin diseases which contains 5-ALA, is sold as a 2-compartment system comprising one compartment with freeze-dried 5-ALA and a solution of 5-ALA is prepared from the 2-compartment system immediately before application.

These approaches, however, have disadvantages. For example, it is not always convenient to transport and store medicines in cold conditions. Moreover it is also generally preferable to provide pharmaceutical compositions in a ready-to-use form as this is most convenient for medical practitioners and staff. Provision of ready-to-use forms also enables the compositions to be prepared with a reliable and accurate concentration. This is particularly important in the treatment and diagnosis of the majority of diseases including cancer where it can be critical that the correct and efficient dosage of therapeutic or diagnostic is administered.

US 2003/125388 describes an alternative approach to provide stable 5-ALA formulations wherein 5-ALA or a derivative thereof is dissolved or dispersed in a non-aqueous liquid having a dielectric constant of less than 80 at 25° C. and wherein said liquid stabilizes 5-ALA or a derivative thereof. It is hypothesized that the use of the non-aqueous liquid facilitates formation of the enol form of 5-ALA that then prevents its degradation. No stability data are shown though. Examples of suitable non-aqueous liquids mentioned in US 2003/125388 include glycerol and its mono-, di- and triesters with C₁-C₂₀ carboxylic acids, propylene glycol, alcohols, ethers, esters, poly(alkylene glycols), phospholipids, DMSO, N-vinylpyrrolidone and N,N-dimethyl acetamide. This composition may form part of a kit for therapeutic or diagnostic use. The other part of the kit is a composition comprising water. In this case the two parts of the kit are mixed prior to use. The approach in US 2003/125388 therefore suffers the same disadvantage as the Levulan Kerastick® in that it is generally undesirable to provide pharmaceutics in a form that requires the medical practitioner to formulate the pharmaceutical product that is actually administered.

The lower part of the gastrointestinal tract, in particular the colon and rectum, may be associated with a number of serious and life-threatening diseases like colitis, colorectal cancer, Crohn's disease, irritable bowel disease and various local infections. Potentially the most serious of these is colorectal cancer. Current diagnostic methods for colorectal cancer include monitoring of clinical symptoms like blood in the stools, lower abdominal pain or weight loss, colonoscopy and X-ray based imaging methods. The prognosis of patients with colorectal cancer depends, as with most other cancer forms, on disease stage at the time of diagnosis and especially on whether the patient has developed distant metastasis. There are several therapeutic drugs in clinical use today for treating colorectal cancer, however, current drugs have their clinical limitations and there remains a medical need for further therapeutic regimes and alternative methods of early diagnosis.

B. Mayinger et al. in Endoscopy 40, 106-109, 2008 describe a clinical study on detection of pre-malignant conditions in the colon by fluorescence endoscopy using enemas comprising 5-ALA hexyl ester dissolved in sterile phosphate buffered saline. The authors show that the use of PDD detects 28% more polyps than when using white light endoscopic imaging.

E. Endlicher et al. in Gastrointestinal Endoscopy 60(3), 449-454, 2004 have used 5-ALA and various 5-ALA esters, i.e. methyl ester, benzyl ester and hexyl ester for the detection of dysplastic lesions by fluorescence in chronic colitis rat models. The 5-ALA and 5-ALA ester derivatives were sterile solutions which were administered as local probes. The sensitivity and specificity in these experiments was dependent on the choice of ester. With 5-ALA hexyl ester, for example, the sensitivity was 60% with a specificity of 51%.

The above-mentioned enemas and locally administered probes have several disadvantages when used for the diagnosis of conditions in the lower part of the gastrointestinal system. These relate to their shelf life stability and form of administration. Both the local administration of the probes as well as the administration of enemas requires the presence of health personnel such as nurses and/or physicians during administration and, in case of the enema, also during incubation. Further, the use of 5-ALA and 5-ALA ester as precursors in PDD requires their conversion into a photosensitiser, i.e. protoporphyrins, which is not an immediate process. Hence there is delay in form of an incubation period between the administration of such a precursor and the light excitation and thus diagnosis. To obtain best diagnostic results, the enema or locally administered probe should be in contact with the colon walls during such an incubation period and it may not be possible for some patients to keep an enema inside their colon for such a period.

Hence there is a need for alternative formulations of 5-ALA and 5-ALA esters and thus pharmaceutical products comprising 5-ALA and 5-ALA ester for use in PDD of the lower part of the gastrointestinal tract, in particular the colon and rectum.

In WO 2009/074811, we describe solid pharmaceutical products for use in PDD of the lower part of the gastrointestinal tract, in particular the colon and rectum. Said solid pharmaceutical products may be for oral administration or in the form of suppositories. Oral solid pharmaceutical products may be in the form of capsules, pellets, powders, tablets, granules, pills or mini-tablets said mini-tablets, powders, granules or pellets may further be provided within a capsule or compressed into a tablet.

We have now surprisingly found new and alternative solid formulations comprising 5-ALA or a derivative thereof (e.g. an ALA ester) for use in PDD of the lower part of the gastrointestinal tract, in particular the colon and rectum.

The new solid formulations have stability at room temperature, are—compared to enema and local probes—easier to handle for the health personnel and provide more convenience for the patients. They can further be readily delivered to the lower part of the gastrointestinal system, especially to the lowest part of the small intestine, the entire colon and rectum. Hence, they overcome the above-mentioned disadvantages of the prior art and are capable of providing an effective concentration of 5-ALA or derivatives thereof at the intended site, i.e. in the lower part of the gastrointestinal tract and, which is important, may also provide a substantially homogenous (i.e. uniform) distribution of 5-ALA or a derivative thereof (e.g. an ALA ester) at said intended site.

Thus, viewed from a first aspect, the invention provides a solid pharmaceutical product for use in the photodynamic diagnosis of cancer, pre-cancerous and non-cancerous conditions in the lower gastrointestinal tract comprising
a) an active ingredient selected from 5-ALA, a precursor of 5-ALA or a derivative of 5-ALA and pharmaceutically acceptable salts thereof;
b) one or more triglycerides; and
c) one or more emulsifiers.

The term “solid” refers to the physical state of the pharmaceutical product, i.e. as being a solid, rather than a liquid or gas. Liquids, dispersions and solutions are therefore not encompassed by this term. Further, semi-solids such as gels, ointments, pastes and creams are neither encompassed by this term. Representative examples of solid pharmaceutical products that are encompassed by the invention include capsules, tablets, pellets, granules, powder and suppositories.

The term “pharmaceutical product” refers to the entity that is actually administered to a subject, e.g. a human or non-human animal.

The term “pre-cancerous condition” denotes a disease, syndrome, or finding that, if left untreated, may lead to cancer. It is a generalized state associated with a significantly increased risk of cancer. A pre-cancerous condition may for instance manifest itself by extensive/abnormal proliferation of cells, e.g. hyperplasia and neoplasia.

The term “non-cancerous conditions” include disease conditions like colitis, Crohn's disease, irritable bowel disease and other viral, bacterial or fungal infections or inflammation located in the lower gastrointestinal tract.

The term “active ingredient” denotes 5-ALA and pharmaceutically acceptable salts thereof, precursors of 5-ALA and pharmaceutically acceptable salts thereof and derivatives of 5-ALA and pharmaceutically acceptable salts thereof.

The term “5-ALA” denotes 5-aminolevulinic acid, i.e. 5-amino-4-oxo-pentanoic acid.

The term “precursor of 5-ALA” denotes compounds which are converted metabolically to 5-ALA and are thus essentially equivalent thereto. Thus the term “precursor of 5-ALA” covers biological precursors for protoporphyrin in the metabolic pathway for haem biosynthesis.

The term “derivative of 5-ALA” includes chemically modified 5-ALA, for example esters.

The term “pharmaceutically acceptable salt” denotes a salt that is suitable for being used in the solid pharmaceutical product and which fulfils the requirements related to for instance safety, bioavailability and tolerability (see for instance P. H. Stahl et al. (eds.) Handbook of Pharmaceutical Salts, Publisher Helvetica Chimica Acta, Zurich, 2002)

The pharmaceutical products of the invention are solid when administered to a subject, e.g. a human or non-human animal. Preferred solid pharmaceutical products of the invention are solid at a temperature of at least 18° C., more preferably at a temperature of at least 25° C., still more preferably at a temperature of at least 30° C.

If the solid pharmaceutical product is not in the form of a suppository, such solid pharmaceutical products of the invention are most preferably solid at a temperature of at least 40° C.

If the solid pharmaceutical product is in the form of a suppository, such solid pharmaceutical products of the invention are most preferably solid at room temperature and will melt/dissolve at body temperature of the subject, i.e. human or non-human animal to whom it is administered to.

In a preferred embodiment, the solid pharmaceutical products according to the invention are for use in photodynamic diagnosis of cancer and pre-cancerous conditions in the lower gastrointestinal tract, preferably in the colon and rectum.

The use of 5-ALA and derivatives thereof, e.g. 5-ALA esters in PDT and PDD is well known in the scientific and patent literature, see, for example, WO 96/28412, WO 2006/051269, WO 2005/092838, WO 03/011265, WO 02/09690, WO 02/10120, WO 2003/041673 and U.S. Pat. No. 6,034,267, the contents of which are incorporated herein by reference. All such derivatives of 5-ALA and their pharmaceutically acceptable salts are suitable for use in the methods herein described.

The synthesis of 5-ALA is known in the art. Further, 5-ALA and pharmaceutically acceptable salts thereof are commercially available, for instance from Sigma Aldrich.

The 5-ALA derivatives useful in accordance with the invention may be any derivative of 5-ALA capable of forming protoporphyrins, e.g. PpIX or PpIX derivatives in vivo. Typically, such derivatives will be a precursor of PpIX or of a PpIX derivative, e.g. a PpIX ester in the biosynthetic pathway for haem and which are therefore capable of inducing an accumulation of PpIX following administration in vivo. Suitable precursors of PpIX or PpIX derivatives include 5-ALA prodrugs which might be able to form 5-ALA in vivo as an intermediate in the biosynthesis of PpIX or which may be converted, e.g. enzymatically, to porphyrins without forming 5-ALA as an intermediate. 5-ALA esters and pharmaceutically acceptable salts thereof are among the preferred compounds for use in the invention described herein.

Esters of 5-ALA which are optionally N-substituted are preferred for use in the invention. Those compounds in which the 5-amino group is unsubstituted, i.e. 5-ALA esters, are particularly preferred. Such compounds are generally known and described in the literature; see, for example, WO 96/28412 and WO 02/10120 to Photocure ASA, the contents of which are incorporated herein by reference.

Esters of 5-ALA with substituted or unsubstituted alkanols, i.e. alkyl esters and substituted alkyl esters, and pharmaceutically acceptable salts thereof, are especially preferred derivatives of 5-ALA for use in the invention. Examples of such compounds include those of general formula I and pharmaceutically acceptable salts thereof:

R²₂N—CH₂COCH₂—CH₂CO—OR¹ (I)

wherein

R¹ represents a substituted or unsubstituted alkyl group; and

R² each independently represents a hydrogen atom or a group R¹

As used herein, the term “alkyl”, unless stated otherwise, includes any long or short chain, cyclic, straight-chained or branched saturated or unsaturated aliphatic hydrocarbon group. Unsaturated alkyl groups may be mono- or polyunsaturated and include both alkenyl and alkynyl groups. Unless stated otherwise, such alkyl groups may contain up to 40 carbon atoms. However, alkyl groups containing up to 30 carbon atoms, preferably up to 10, particularly preferably up to 8, especially preferably up to 6 carbon atoms are preferred.

In compounds of formula I, the R¹ groups are substituted or unsubstituted alkyl groups. If R¹ is a substituted alkyl group, one or more substituents are either attached to the alkyl group and/or interrupt the alkyl group. Suitable substituents that are attached to the alkyl group are those selected from hydroxy, alkoxy, acyloxy, alkoxycarbonyloxy, amino, aryl, nitro, oxo, fluoro, —SR₃, —NR³₂ and —PR³₂, wherein R³ is a hydrogen atom or a C₁₋₆ alkyl group. Suitable substituents that interrupt the alkyl group are those selected from —O—, —NR₃—, —S— or —PR₃.

In a preferred embodiment, R¹ is an alkyl group substituted with one or more aryl substituents, i.e. aryl groups, preferably substituted with one aryl group.

As used herein, the term “aryl group” denotes an aromatic group which may or may not contain heteroatoms like nitrogen, oxygen or sulfur. Aryl groups which do not contain heteroatoms are preferred. Preferred aryl groups comprise up to 20 carbon atoms, more preferably up to 12 carbon atoms, for example, 10 or 6 carbon atoms. Preferred embodiments of aryl groups are phenyl and naphthyl, especially phenyl. Further, the aryl group may optionally be substituted by one or more, more preferably one or two, substituents. Preferably, the aryl group is substituted at the meta or para position, most preferably the para position. Suitable substituents include halo alkyl, e.g. trifluoromethyl, alkoxy, preferably alkoxy groups containing 1 to 6 carbon atoms, halo, e.g. iodo, bromo, chloro or fluoro, preferably chloro and fluoro, nitro and C₁₋₆ alkyl, preferably C₁₋₄ alkyl. Preferred C₁₋₆ alkyl groups include methyl, isopropyl and t-butyl, particularly methyl. Particularly preferred aryl substituents are chloro and nitro. However, still more preferably the aryl group is unsubstituted.

Preferred such aryl substituted R¹ groups are benzyl, 4-isopropylbenzyl, 4-methylbenzyl, 2-methylbenzyl, 3-methylbenzyl, 4-[t-butyl]benzyl, 4-[trifluoromethyl]benzyl, 4-methoxybenzyl, 3,4-[di-chloro]benzyl, 4-chlorobenzyl, 4-fluorobenzyl, 2-fluorobenzyl, 3-fluorobenzyl, 2,3,4,5,6-pentafluorobenzyl, 3-nitrobenzyl, 4-nitrobenzyl, 2-phenylethyl, 4-phenylbutyl, 3-pyridinyl-methyl, 4-diphenyl-methyl and benzyl-5-[(1-acetyloxyethoxy)-carbonyl]. More preferred such R1 groups are benzyl, 4-isopropylbenzyl, 4-methylbenzyl 4-nitrobenzyl and 4-chlorobenzyl. Most preferred is benzyl.

If R¹ is a substituted alkyl group, one or more oxo substituents are preferred. Preferably, such groups are straight-chained C₄₋₁₂ alkyl groups which are substituted by one or more oxo groups, preferably by one to five oxo groups. The oxo groups are preferably present in the substituted alkyl group in an alternating order, i.e. resulting in short polyethylene glycol substituents. Preferred examples of such groups include 3,6-dioxa-1-octyl and 3,6,9-trioxa-1-decyl.

If R¹ is an unsubstituted alkyl group, R¹ groups that are saturated straight-chained or branched alkyl groups are preferred. If R¹ is a saturated straight-chained alkyl group, C₁₋₁₀ straight-chained alkyl group are preferred. Representative examples of suitable straight-chained alkyl groups include methyl, ethyl, n-propyl, n-butyl, n-pentyl, n-hexyl and n-octyl. Particularly preferred are C₁₋₆ straight-chained alkyl group, most particularly preferred are methyl and n-hexyl. If R¹ is a saturated branched alkyl group, such branched alkyl groups preferably consist of a stem of 4 to 8, preferably 5 to 8 straight-chained carbon atoms and said stem is branched by one or more C₁₋₆ alkyl groups, preferably C₁₋₂ alkyl groups. Examples of such saturated branched alkyl groups include 2-methylpentyl, 4-methylpentyl, 1-ethylbutyl and 3,3-dimethyl-1-butyl.

In compounds of formula I, each R² independently represents a hydrogen atom or a group R¹. Particularly preferred for use in the invention are those compounds of formula I in which at least one R² represents a hydrogen atom. In especially preferred compounds each R² represents a hydrogen atom.

Preferably, compounds of formula I and pharmaceutically acceptable salts thereof are used in the solid pharmaceutical product of the invention, wherein R¹ is methyl or hexyl, more preferably n-hexyl and both R² represent hydrogen, i.e. 5-ALA methyl ester, 5-ALA hexyl ester and pharmaceutically acceptable salts thereof, preferably the HCl salts. The preferred compound for use in the solid pharmaceutical product of the invention is 5-ALA hexyl ester and pharmaceutically acceptable salts thereof, preferably the HCl salt or sulfonic acid salts or sulfonic acid derivative salts.

5-ALA esters and pharmaceutically acceptable salts thereof for use in the invention may be prepared by any conventional procedure available in the art, e.g. as described in WO 96/28412 and WO 02/10120. Briefly, 5-ALA esters may be prepared by reaction of 5-ALA with the appropriate alcohol in the presence of a catalyst, e.g. an acid. Pharmaceutically acceptable salts of 5-ALA esters may be prepared as described before by reaction of a pharmaceutically acceptable 5-ALA salt, e.g. 5-ALA hydrochloride with the appropriate alcohol. Alternatively compounds for use in the invention like 5-ALA methyl ester or 5-ALA hexyl ester may be available commercially, e.g. from Photocure ASA, Norway.

The 5-ALA esters for use in the invention may be in the form of a free amine, e.g. —NH₂, —NHR² or —NR²R² or preferably in the form of a pharmaceutically acceptable salt. Such salts preferably are acid addition salts with pharmaceutically acceptable organic or inorganic acids. Suitable acids include, for example, hydrochloric, nitric, hydrobromic, phosphoric, sulfuric, sulfonic and sulfonic acid derivatives, the latter are described in WO2005/092838 to Photocure ASA, the entire contents of which are incorporated herein by reference. A preferred acid is hydrochloride acid, HCl, sulfonic acid and sulfonic acid derivatives. Procedures for salt formation are conventional in the art.

Thus, a preferred embodiment of the invention is a solid pharmaceutical product for use in the photodynamic diagnosis of cancer, pre-cancerous and non-cancerous conditions in the lower gastrointestinal tract comprising
a) a derivative of 5-ALA or a pharmaceutically salt thereof, preferably a 5-ALA ester or a pharmaceutically acceptable salt thereof;
b) one or more triglycerides; and
c) one or more emulsifiers.

In a preferred embodiment, said 5-ALA ester is a compound of formula (I) or an pharmaceutically acceptable salt thereof, wherein R¹ represents an unsubstituted alkyl group, preferably an unsubstituted saturated straight-chained or branched alkyl group, more preferably an unsubstituted saturated straight-chained C₁₋₁₀ alkyl group. More preferably, said 5-ALA ester is 5-ALA hexyl ester and in a further preferred embodiment, said pharmaceutically acceptable salt of 5-ALA hexyl ester is the HCl salt or a sulfonic acid salt or sulfonic acid derivative salt, such as mesylate, tosylate or napsylate.

The compounds hereinbefore described may be used for the manufacture of the solid pharmaceutical product according to the invention in any conventional manner. The desired concentration of 5-ALA or derivative of 5-ALA or precursor of 5-ALA in the pharmaceutical products of the invention will vary depending on several factors including the nature of the compound, the nature and form of the product in which this is presented, the intended mode of administration and the subject, i.e. the human or non-human animal, to be treated. Generally, however, the concentration of 5-ALA or derivative of 5-ALA or precursor of 5-ALA or pharmaceutically acceptable salts thereof is conveniently in the range 1 to 50%, preferably 1 to 40%, e.g. 2 to 35%, more preferably 5 to 30% by weight of the total weight of the sum of ingredients a) plus b) plus c).

The solid pharmaceutical products according to the invention comprise one or more triglycerides, i.e. triacylglycerols. The triglyceride is comprised of one molecule glycerol and 3 fatty acid molecules. The 3 fatty acids may be identical or different fatty acids.

The triglycerides may be solid or liquid at room temperature, i.e. at temperatures of about 18° C. to about 25° C. Solid triglycerides are commonly denoted fat, while liquid triglycerides are commonly denoted oil. If solid triglycerides are used, said solid triglycerides have preferably a melting point of below or at the body temperature of the human or non-human animal the solid pharmaceutical product is administered to. In a preferred embodiment the solid pharmaceutical product is administered to a human and the melting point of a solid triglyceride comprised in said pharmaceutical product is between about 26° C. and 37° C.

The triglycerides may be synthetic, semi-synthetic or of animal and/or vegetable origin. The triglycerides may be pure/isolated triglycerides or a part of a mixture, such as a mixture of triglycerides, monoglycerides and/or diglycerides and/or free fatty acids and/or unsaponifiable lipids. Such mixtures are typically found edible oils of animal and/or vegetable origin. If the triglycerides are part of a mixture, they preferably constitute the major part of said mixture. In the following, such mixtures are also denoted “triglycerides”.

Since the triglycerides are used in the pharmaceutical product according to the invention, which will be used in a human or non-human animal, they need to be of pharmaceutical grade and fulfill the requirements and standards of such products with regard to physiological acceptance, tolerability and safety.

Further, the triglycerides should be inert compounds, i.e. compounds which do not react with the active ingredient a) or which do not promote degradation of the active ingredient.

The term “one or more triglycerides” means that the solid pharmaceutical product according to the invention contains one triglyceride or several different triglycerides. By way of example, the solid pharmaceutical product may contain tricaprylin (caprylic acid triglyceride) or tricaprylin and caprylic/capric triglyceride. Further, by way of example the solid pharmaceutical product may contain soybean oil, which is a mixture of triglycerides of alpha-linolenic acid, linoleic acid, oleic acid, stearic acid and palmitic acid.

Preferred triglycerides are selected from edible oils of animal and/or vegetable origin and/or fractions thereof, such as soybean oil, palm oil, palm kernel oil, corn oil, olive oil, almond oil, safflower oil, peanut oil, coconut oil, sunflower oil, castor oil, pine oil, jojoba oil, cocoa butter, and palm olein. Further examples of preferred triglycerides are illipe butter, shea butter, cocoa butter, kokum butter, sal butter and other natural oils or fractions thereof. Other examples of preferred triglycerides include hydrogenated or partially hydrogenated triglycerides selected from partially or fully hydrogenated soybean oil, rapeseed oil, cottonseed oil, sunflower oil, coconut oil and fractions thereof. The triglycerides may also be synthetic or semi-synthetic triglycerides, such as medium-chain triglycerides (MCT).

In a preferred embodiment, the triglyceride is a triglyceride of glycerol and 3 identical or different C₂-C₂₂ fatty acids, more preferably 3 identical or different C₄-C₁₈ fatty acids, even more preferably 3 identical or different C₆-C₁₈ fatty acids and most preferably 3 identical or different C₆-C₁₂ fatty acids. In a more preferred embodiment, the triglyceride is a triglyceride of glycerol and 3 identical C₂-C₂₂ fatty acids, more preferably 3 identical C₄-C₁₈ fatty acids, even more preferably 3 identical C₆-C₁₈ fatty acids and most preferably 3 identical C₆-C₁₂ fatty acids.

Most preferred solid triglycerides are cocoa butter, tallow, hard fat, hydrogenated coco-glycerides, hydrogenated palm oil, tristearin, tripalmitin and trimyristin. Such solid triglycerides are especially preferred if the solid pharmaceutical product is a suppository. For suppositories, hydrogenated coco-glycerides, optionally mixed with glyceryl ricinoleate, e.g. those marketed under the name “Witepsol®” and “Massa Estarinum®” are preferred, more preferably those hydrogenated coco-glycerides with a low hydroxyl value and a melting point between 31° C. and 38° C., i.e. Witepsol H 32, Witepsol H 35, Witepsol H 37 and Massa Estarinum® 299.

Most preferred liquid triglycerides are tricaprylin, tricaproin, triheptanoin, caprylic/capric triglyceride, caprylic/capric/linoleic triglyceride and caprylic/capric/succinic triglyceride, some of these liquid triglycerides are marketed under the name “Miglyol®”, e.g. with Miglyol 812 being caprylic/capric triglyceride, Miglyol 818 being caprylic/capric/linoleic triglyceride and Miglyol 808 being tricaprylin. A manufacturer or such triglycerides is for instance Sasol, Witten, Germany.

Generally, the amount of triglyceride in the pharmaceutical product of the invention is 50 to 90%, more preferably 60 to 80% by weight of the total weight of the sum of ingredients a) plus b) plus c).

The triglycerides used in the invention may be prepared using standard processes and procedures well-known in the art, and are generally commercially available from various manufacturers such like Sasol, Croda, Cognis, Gattefossé and others.

The solid pharmaceutical products according to the invention comprise one or more emulsifiers.

An emulsifier, also known as a surfactant, surface active material or emulgent, is a substance which stabilizes an emulsion. A wide variety of emulsifiers are used to prepare emulsions which can be used in a pharmaceutical product.

The term “one or more emulsifier” means that the solid pharmaceutical product according to the invention contains one emulsifier or several different emulsifiers.

The emulsifier used in the solid pharmaceutical product of the invention may be solid or liquid at room temperature, i.e. at temperatures of about 18° C. to about 25° C.

In a preferred embodiment, emulsifiers are non-ionic emulsifiers.

Preferred non-ionic emulsifiers are selected from the group of short chain partial glycerides, i.e. esters of glycerol with short chain fatty acids, whereby only a part of the existing hydroxyl groups are esterified, i.e. mono- or diglycerides or mixtures of mono- and diglycerides. Preferred partial glycerides are mono- or diglycerides or mixtures of mono- and diglycerides of C₆-C₁₀ fatty acids.

Yet other preferred non-ionic emulsifiers are esters of glycerol with fatty acids and alpha-hydroxy acids, for instance glyceryl stearate citrate, glyceryl citrate/lactate/oleate/linoleate, glyceryl cocoate/citrate/lactate and glyceryl isostearate.

Yet other preferred non-ionic emulsifiers are fatty alcohols and/or ethoxylated fatty alcohols like cetostearyl alcohol or cetomacrogol.

Yet other preferred non-ionic emulsifiers are ethoxylated fatty acids, like ethoxylated castor oil.

Yet other preferred non-ionic emulsifiers are non-ethoxylated and ethoxylated esters of sorbitan and fatty acids, sold under the name “Span” and “Tween”, i.e. polysorbates, preferably (polyoxyethylene) sorbitan monolaurate, (polyoxyethylene) sorbitan monopalmitate, (polyoxyethylene) sorbitan monostearate, (polyoxyethylene) sorbitan monooleate, (polyoxyethylene) sorbitan tristearate or (polyoxyethylene) sorbitan trioleate.

Yet other preferred non-ionic emulsifiers are lecithins, e. g. egg yolk lecithin or soybean lecithin or phospholipids derived from lecithin, preferably phosphatidylcholine.

Yet other preferred non-ionic emulsifiers are polyethylene glycol based compounds like polyethylene glycol 400 monostearate.

Yet other preferred non-ionic emulsifiers are ethoxylated glycerides, like ethoxylated caprylocaproyl glyceride or products obtained from the reaction of polyethylene glycol and natural or hydrogenated oils, such as palm kernel oil, hydrogenated palm kernel oil, castor oil, hydrogenated castor oil, almond oil, apricot kernel oil and the like.

These latter non-ionic emulsifiers are more preferred and preferred examples are lauroyl macrogol-32 glyceride, Gelucire® 44/14 (Gattefossé); stearoyl macrogol glyceride, Gelucire® 50/13 (Gattefossé); PEG-50 castor oil, Emalex C-50 (Nihon Emulsion); Eumulgin® HRE 40 (Cognis); PEG-45 hydrogenated castor oil, PEG-8 caprylic/capric glycerides, Labrasol® (Gattefossé); either alone or in a mixture with other emulsifiers. In a preferred embodiment, several Gelucires are mixed such as instance Gelucire® 44/14 with Gelucire® 50/02 (saturated polyglycolized glycerides), or Gelucire® 33/01 (glycerol esters of C8-C18 saturated fatty acids).

Yet other more preferred non-ionic emulsifiers are poloxamers, i.e. triblock copolymers composed of a central hydrophobic chain of polyoxypropylene flanked by two hydrophilic chains of polyoxyethylene. Poloxamers are also known by the trade name Pluronics®. Most preferred poloxamers are those which are liquid and have a pH below 7, preferably below 6 such as Pluronic® L43, HLB 7-12 and Pluronic® L44, HLB 12-18, either alone or in a mixture with other emulsifiers, preferably other poloxamers such as Pluronic® F68.

If the active ingredient a) is a C₁-C₁₀ alkyl ester of 5-ALA or a pharmaceutically acceptable salt thereof, preferably non-ionic emulsifiers with high hydrophilic-lipophilic balance values (HLB values) are used, even more preferably with a HLB value of at least 7, preferably with a HLB value of at least 12, more preferably with an HLB value of about 12-18. If more than one emulsifier is used, it is also possible to use an emulsifier with an HLB value below 7 or above 18, provided that the resulting mixture of emulsifiers has a HLB value of at least 7 and preferably a HLB value of about 12-18.

Generally, the emulsifier is present in the solid pharmaceutical product in an amount necessary to promote uniform distribution of the pharmaceutical product at the site of use, e.g. in the colon and rectum. Suitably, amount of the emulsifier is chosen in view of the amount of triglycerides. Preferably, the emulsifier is present in the pharmaceutical product of the invention, in an amount of about 0.5 to 50%, preferably 1 to 35%, more preferably 2 to 30% by weight of the total weight of the solid pharmaceutical product.

The emulsifiers used in the invention may be prepared using standard processes and procedures well-known in the art, although many are available commercially from various manufacturers such like Sasol, Croda, Cognis, Gattefossé, American Lecithin Company, BASF, Cytec and others.

The solid pharmaceutical product further comprises
d) optionally one or more mucoadhesives
e) optionally one or more pharmaceutically acceptable excipients other than b), and c);
f) optionally one or more surface penetration agents; and
g) optionally one or more chelating agents.

The solid pharmaceutical product according to the invention optionally comprises one or more mucoadhesives, i.e. one mucoadhesive or several different mucoadhesives.

The term “mucoadhesive” denotes a compound which exhibits an affinity for a mucosa surface, i.e. adhere to that surface through the formation of bonds which are generally non-covalent in nature, whether binding occurs through interaction with the mucous and/or the underlying cells. In the context of the invention, the mucosa surface is a mucosa surface of the lower gastrointestinal tract, in particular the mucosa of the colon and the rectum.

The mucoadhesive which is optionally present in the solid pharmaceutical product of the invention is preferably a mucoadhesive that is not degraded or metabolized by bacterial and non-bacterial enzymes present in the lower gastrointestinal tract, in particular in the colon and the rectum.

Mucoadhesives which may be used in the solid pharmaceutical products of the invention may be natural or synthetic compounds, polyanionic, polycationic or neutral, water-soluble or water-insoluble, but are preferably large, e. g. having a molecular weight of 500 kDa to 3000 kDa, e. g. 1000 kDa to 2000 kDa, water-insoluble cross-linked, e. g. containing 0.05% to 2% cross-linker by weight of the total polymer, prior to any hydration, water-swellable polymers capable of forming hydrogen bonds. Preferably such mucoadhesive compounds have a mucoadhesive force greater than 100, especially preferably greater than 120, particularly greater than 150, expressed as a percent relative to a standard in vitro, as assessed according to the method of Smart et al., 1984, J. Pharm. Pharmacol., 36, pp 295-299.

Preferred mucoadhesives are selected from polysaccharides, preferably dextran, pectin, amylopectin or agar; gums, preferably guar gum or locust bean gum; salts of alginic acid, preferably sodium alginate or magnesium alginate; poly(acrylic acid) and crosslinked or non-crosslinked copolymers of poly(acrylic acid) and derivatives of poly(acrylic acid) such as salts and esters like for instance carbomer (carbopol).

When present, the mucoadhesives may conveniently be provided in a concentration range of 0.05 to 50%, preferably 0.1 to 25%, e.g. 0.2 to 10% by weight of the total weight of the solid pharmaceutical product according to the invention.

The solid pharmaceutical product according to the invention optionally comprises one or more pharmaceutically acceptable excipients which are different from the excipients b), c) and the optional excipient d). Such optional one or more pharmaceutically acceptable excipients may be selected from the group of antiadherents, fillers, binders, flavors, colors, odor enhancers, glidants, lubricants, disintegrants, solvents or preservatives. The skilled man will be able to select suitable excipients based on, for example, the route of administration chosen. Excipients that may be used in the pharmaceutical products herein described are listed in various handbooks (e.g. D. E. Bugay and W. P. Findlay (Eds) Pharmaceutical excipients (Marcel Dekker, New York, 1999), E-M Hoepfner, A. Reng and P. C. Schmidt (Eds) Fiedler Encyclopedia of Excipients for Pharmaceuticals, Cosmetics and Related Areas (Edition Cantor, Munich, 2002) and H. P. Fielder (Ed) Lexikon der Hilfsstoffe für Pharmazie, Kosmetik and angrenzende Gebiete (Edition Cantor Aulendorf, 1989)).

If the solid pharmaceutical product according to the invention optionally comprises one or more pharmaceutically acceptable solvents, such solvents may be a free fatty acid, a free fatty alcohol, an aqueous solution, e.g. a buffer, or water. However, it is preferred that the solid pharmaceutical product according to the invention does not contain any water, i.e. is water-free. By water-free, it is meant that no water is added to the solid pharmaceutical product and that any measurable water content of the product is due to water possibly contained in any of the ingredients a)-g).

The solid pharmaceutical product according to the invention optionally comprises one or more surface penetration assisting agents. Such agents may have a beneficial effect in enhancing the photosensitizing effect of 5-ALA, the derivative of 5-ALA or the precursor of 5-ALA present in the pharmaceutical products of the invention.

Surface penetration assisting agents, especially dialkylsulphoxides such as dimethylsulphoxide (DMSO) may therefore be included in the products. The surface penetration assisting agent may be any of the skin penetration assisting agents described in the pharmaceutical literature, e.g. chelators (e.g. EDTA), surfactants (e.g. sodium dodecyl sulfate), non-surfactants, bile salts (sodium deoxycholate) and fatty acids (e.g. oleic acid). Examples of appropriate surface penetration assisting agents include isopropanol, 1-[2-(decylthio)ethyl]-azacyclopentan-2-one (HPE-101 available from Hisamitsu), DMSO and other dialkylsulphoxides, in particular n-decylmethyl sulphoxide (NDMS), dimethylsulphacetamide, dimethylformamide (DMFA), dimethylacetamide, isopropylmyristate, oleylalcohol and oleic acid, various pyrrolidone derivatives (Woodford et al., J. Toxicol. Cut. & Ocular Toxicology, 1986, 5: 167-177) and Azone® (Stoughton et al., Drug Dpv. Ind. Pharm. 1983, 9: 725-744) or mixtures thereof.

The use of glycols such as propylene glycol as surface penetration assisting agents is not recommended since it may promote the degradation of the active ingredient a) in the solid pharmaceutical product according to the invention.

The surface penetration assisting agent may conveniently be provided in a concentration range of 0.2 to 50% by weight of the total weight of the pharmaceutical product in which it is present, e.g. about 0.5 to 5% by weight of the total weight of the solid pharmaceutical product in which it is present.

The solid pharmaceutical product according to the invention optionally comprises one or more chelating agents. Such agents may also have a beneficial effect in enhancing the photosensitizing effect of 5-ALA, the derivative of 5-ALA or the precursor of 5-ALA present in the pharmaceutical products of the invention.

Chelating agents may, for example, be included in order to enhance the accumulation of PpIX since the chelation of iron by the chelating agent prevents its incorporation into PpIX to form haem by the action of the enzyme ferrochelatase, thereby leading to a build up of PpIX. The photosensitizing effect is therefore enhanced.

Suitable chelating agents that may be included in the solid pharmaceutical product aminopolycarboxylic acids, such as any of the chelants described in the literature for metal detoxification or for the chelation of paramagnetic metal ions in magnetic resonance imaging contrast agents. Particular mention may be made of EDTA, CDTA (cyclohexane triamine tetraacetic acid), DTPA and DOTA and well known derivatives and analogues thereof. EDTA and DTPA are particularly preferred. Other suitable chelating agents are desferrioxamine and siderophores and they may be used alone or in conjunction with aminopolycarboxylic acid chelating agents such as EDTA.

Some of the above-mentioned chelating agents do also exhibit surface penetration assisting agent properties, e.g. EDTA.

Where present, the chelating agent may conveniently be used at a concentration of 0.01 to 12%, e.g. 0.1 to 10% by weight of the total weight of the solid pharmaceutical product.

The solid pharmaceutical products according to the invention are either for oral or rectal administration, preferably for oral administration.

For rectal administration (rectal insertion), the solid pharmaceutical product according to the invention is preferably provided in form of a suppository.

Preferably, a solid pharmaceutical product according to the invention which is provided in the form of a suppository (hereinafter denoted “suppository of the invention”) comprises as one or more triglycerides b) one or more solid triglycerides having a melting point of below or at the body temperature of the human or non-human animal the suppository is administered to. In a preferred embodiment the suppository is administered to a human and the melting point of said one or more solid triglycerides is between about 26° C. and 37° C. Preferred such solid triglycerides are cocoa butter, tallow, hard fat, hydrogenated coco-glycerides, more preferably hydrogenated coco-glycerides, e.g. those marketed under the name “Witepsol®” and “Massa Estarinum®” (e.g. by Sasol), even more preferably those hydrogenated coco-glycerides with a low hydroxyl value and a melting point between 31° C. and 38° C., i.e. Witepsol® H 32, Witepsol® H 35, Witepsol® H 37 and Massa Estarinum® 299. Solid triglycerides which have a melting point above body temperature, e.g. hydrogenated palm oil, tristearin, tripalmitin or trimyristin, may be used in a mixture with liquid or solid triglycerides, as long as the melting point of said mixture is between about 26° C. and 37° C.

Preferably, a solid pharmaceutical product according to the invention which is provided in the form of a suppository (hereinafter denoted “suppository of the invention”) comprises as one or more emulsifiers lecithin, phosphatidylcholine, poloxamers, ethoxylated fatty alcohols or products obtained from the reaction of polyethylene glycol and natural or hydrogenated oils.

The suppository may be prepared by any conventional method, e.g. by direct compression of the compounds a)-c) and optionally d)-h), by compression after granulation or by molding, e.g. by melting the one or more solid triglycerides, mixing them with the active ingredient, the one or more emulsifiers and optional other compounds and pouring the mixture into a casting mould where it cools and hardens.

If the solid pharmaceutical product is in the form of a suppository for rectal administration, the active ingredient needs to be released from the suppository at or slightly below body temperature of the subject, e.g. human or non-human animal, the suppository is administered to. Thus, preferred solid pharmaceutical product which are in the form of a suppository are solid at a temperature of below the body temperature of the subject they are administered to, more they are solid at a temperature of at least 30° C. and melt at temperatures above, e.g. in the range of 31° C. to about 42° C. If the subject is a human, they preferably melt in the range of about 31° C. to about 37° C.

For oral administration, the solid pharmaceutical product according to the invention is provided in conventional solid form, e.g. powder, granule, pellet, tablet or capsule, whereby said capsules contain the ingredients a)-c) and optional ingredients d)-g) in the form of powder, granules, pellets, mini-tablets or as a semi-solid or liquid.

In one preferred embodiment, a solid pharmaceutical product according to the invention which is provided in the form of a capsule comprises as one or more triglycerides b) one or more liquid triglycerides, preferably selected from triglycerides of glycerol and 3 identical or different C₂-C₂₂ fatty acids, more preferably 3 identical or different C₄-C₁₈ fatty acids, even more preferably 3 identical or different C₆-C₁₈ fatty acids and most preferably 3 identical or different C₆-C₁₂ fatty acids, more preferably tricaprylin, tricaproin, triheptanoin, caprylic/capric triglyceride, caprylic/capric/linoleic triglyceride and most preferably caprylic/capric triglyceride. To prepare the solid pharmaceutical product, i.e. to fill the capsule, the one or more liquid triglycerides may be mixed with the active ingredient, together with the one or more emulsifiers and optional other compounds d)-g). Preferred emulsifiers are lecithins, phosphatidylcholine, ethoxylated glycerides, polyoxyethylene sorbitan monooleate, dioctyl sodium sulfosuccinate, sodium lauryl sulfate, poloxamers and products obtained from the reaction of polyethylene glycol and natural or hydrogenated oils.

In another preferred embodiment, a solid pharmaceutical product according to the invention which is provided in the form of a capsule comprises as one or more triglycerides b) one or more solid triglycerides having a melting point of below or at the body temperature of the human or non-human animal the capsule is administered to. In a preferred embodiment the capsule is administered to a human and the melting point of said one or more solid triglycerides is between about 26° C. and 37° C. Preferred such solid triglycerides are cocoa butter, tallow, hard fat, hydrogenated coco-glycerides, hydrogenated palm oil, tristearin, tripalmitin or trimyristin, more preferably hydrogenated coco-glycerides optionally mixed with glyceryl ricinoleate, e.g. those marketed under the name “Witepsol®” and “Massa Estarinum®”, even more preferably those hydrogenated coco-glycerides with a low hydroxyl value and a melting point between 31° C. and 38° C., i.e. Witepsol® H 32, Witepsol® H 35, Witepsol® H 37 and Massa Estarinum® 299. To prepare the solid pharmaceutical product, i.e. to fill the capsule, the one or more solid triglycerides may be melted and the active ingredient is mixed with the melted triglycerides, together with the one or more emulsifiers and optional other compounds d)-g). Preferred emulsifiers selected from lecithins, phosphatidylcholine, ethoxylated glycerides, polyoxyethylene sorbitan monooleate, dioctyl sodium sulfosuccinate, sodium lauryl sulfate, poloxamers and products obtained from the reaction of polyethylene glycol and natural or hydrogenated oils.

In yet another preferred embodiment, a solid pharmaceutical product according to the invention which is provided in the form of a capsule comprises several triglycerides b) which are liquid and solid, e.g. one solid triglyceride and one liquid triglyceride. The solid triglycerides have a melting point of below or at the body temperature of the human or non-human animal the capsule is administered to. In a preferred embodiment the capsule is administered to a human and the melting point of said solid triglycerides is between about 26° C. and 37° C. Preferred liquid triglycerides are selected from triglycerides of glycerol and 3 identical or different C₂-C₂₂ fatty acids, more preferably 3 identical or different C₄-C₁₈ fatty acids, even more preferably 3 identical or different C₆-C₁₈ fatty acids and most preferably 3 identical or different C₆-C₁₂ fatty acids, more preferably tricaprylin, tricaproin, triheptanoin, caprylic/capric triglyceride, caprylic/capric/linoleic triglyceride and most preferably caprylic/capric triglyceride. Preferred solid triglycerides are cocoa butter, tallow, hard fat, hydrogenated coco-glycerides, hydrogenated palm oil, tristearin, tripalmitin or trimyristin, more preferably hydrogenated coco-glycerides optionally mixed with glyceryl ricinoleate, e.g. those marketed under the name “Witepsol®” and “Massa Estarinum®”, even more preferably those hydrogenated coco-glycerides with a low hydroxyl value and a melting point between 31° C. and 38° C., i.e. Witepsol® H 32, Witepsol® H 35, Witepsol® H 37 and Massa Estarinum® 299. To prepare the solid pharmaceutical product, i.e. to fill the capsule, the one or more solid triglycerides may be melted and mixed with the one or more liquid triglycerides, the active ingredient, the one or more emulsifiers and optional other compounds d)-g). Preferred emulsifiers are selected from lecithins, phosphatidylcholine, ethoxylated glycerides, polyoxyethylene sorbitan monooleate, dioctyl sodium sulfosuccinate and sodium lauryl sulfate, poloxamers, products obtained from the reaction of polyethylene glycol and natural or hydrogenated oils and ethoxylated fatty alcohols.

In a more preferred embodiment, a solid pharmaceutical product according to the invention which is provided in the form of a capsule comprises liquid triglycerides b) selected from triglycerides of glycerol and 3 identical or different C₂-C₂₂ fatty acids, more preferably 3 identical or different C₄-C₁₈ fatty acids, even more preferably 3 identical or different C₆-C₁₈ fatty acids and most preferably 3 identical or different C₆-C₁₂ fatty acids, more preferably tricaprylin, tricaproin, triheptanoin, caprylic/capric triglyceride, caprylic/capric/linoleic triglyceride and most preferably caprylic/capric triglyceride and a non-ionic emulsifier, the emulsifier being preferably a poloxamer or a product obtained from the reaction of polyethylene glycol and natural or hydrogenated oils, more preferably Pluronic® L43, Pluronic® L44, lauroyl macrogol-32 glyceride, Gelucire® 44/14 (Gattefosse); or stearoyl macrogol glyceride, Gelucire® 50/13 (Gattefosse). To prepare the solid pharmaceutical product, i.e. to fill the capsule, the one or more solid triglycerides may be melted and mixed with the one or more liquid triglycerides, the active ingredient, the one or more emulsifiers and optional other compounds d)-g). Alternatively, the liquid triglyceride, emulsifier and optional other compounds d)-g) may be formed to pellets, mini-tablets or granules, and excipients known in the art to form such pellets, mini-tablets or granules may be added, such as viscosity enhancers or fillers. The so-formed pellets, mini-tablets or granules are then filled into a capsule.

If for oral administration, the solid pharmaceutical product according to the invention is provided in the form of powder, granules, tablets, pellets, capsules or mini-tablets, said products comprise as the one or more triglycerides solid and/or liquid triglycerides. Tablets, powder, granules, pellets or mini-tablets may be prepared by any conventional method. Preferably, tablets and mini-tablets are prepared by direct compression of the compounds a)-c) and optionally d)-g) or by compression after granulation.

Since the oral solid pharmaceutical product is for use in the photodynamic treatment or diagnosis of cancer, pre-cancerous and non-cancerous conditions in the lower part of the gastrointestinal system, the pharmaceutical product needs to reach the lower part of the gastrointestinal system intact, i.e. without the active ingredient being released earlier. To achieve successful delivery to the lower gastrointestinal tract, the active ingredient needs to be protected from absorption and/or the environment of the upper gastrointestinal tract, e.g. stomach and upper small intestine and then be released into the lower gastrointestinal tract, i.e. the lower end of the small intestine and caecum. It is an advantage that the active ingredient is substantially homogenously (i.e. uniformly) distributed to the whole lower gastrointestinal tract. This requires release of the active ingredient into the lower gastrointestinal tract, i.e. the lower end of the small intestine and caecum but also the distribution/spreading of the active ingredient from the place of release to the more distal parts of the colon and the rectum. This may be achieved by delayed release, i.e. the release of the active ingredient starts at the lower end of the small intestine and caecum and is delayed rather than abrupt, such that the pharmaceutical product can travel through the colon with the active ingredient being released by and by. In another embodiment, this may be achieved by using two or more oral solid pharmaceutical products according to the invention with different release profiles or one oral solid pharmaceutical product, e.g. a capsule, comprising e.g. mini-tablets, pellets or granules with different release profiles.

There are various known methods and systems for oral colonic delivery of pharmaceutical active ingredients which are based on that an oral pharmaceutical product comprises one or more pharmaceutical excipients that provide for controlled release of the active ingredient and/or by coating the oral pharmaceutical product with a coating that provides such a time controlled release.

Pressure-controlled systems utilize the increase in pressure of the luminal contents to effect release of the active ingredient. In one embodiment, the active ingredient is dispersed in a melted solid triglyceride (suppository base) which will melt at body temperature, together with one or more emulsifier and the mixture is cooled such that a solid pharmaceutical product according to the invention is obtained. The solid pharmaceutical product is coated with ethyl cellulose. After the product has been swallowed, temperature of body is responsible for the suppository base to melt which increases the volume within the coating such that a balloon is formed of ethyl cellulose filled with liquid. This balloon is capable of remaining intact in the small intestine but will rupture when exposed to the more intense contractions and luminal contents of higher viscosity encountered in the colon.

Time-controlled systems (pulsatile release systems) are based on the principle of delaying the time of drug release until the system transits from mouth to colon Pulsatile release systems are formulated to undergo a lag-time of predetermined span of time of no release, followed by a rapid and complete release or a delayed release of the loaded drugs(s). A lag-time of 5 hours is usually considered sufficient since small intestine transit is about 3-4 hours, which is relatively constant and hardly affected by the nature of formulation administered. In one embodiment, the oral solid pharmaceutical product is coated with lipid barriers such as carnauba wax and/or beeswax along with surfactants like polyoxyethylene sorbitan monooleate. When the product comes in contact with aqueous medium the coat emulsifies or erodes after the lag-time depending on the thickness of coat. The lag time of this system is independent of the gastrointestinal motility, pH, enzyme and gastric residence time. In another embodiment, the pharmaceutical product (liquid or solid) is filled into an insoluble capsule body housing which is sealed with a plug of a swellable hydrogel. Upon contact with gastrointestinal fluid the plug swells pushing itself out of the capsule after the lag-time, which is controlled by the position and the dimensions of the plug. The plug material may be made up of (i) swellable materials coated with insoluble but permeable polymer, e.g. polymethacrylates; (ii) erodible compressed polymer like HPMC, polyvinyl alcohol, polyethylene oxide; (iii) congealed melted polymer like glyceryl monooleate or enzymatically controlled erodible polymer like pectin. In a preferred embodiment and to account for variable gastric residence time the capsule is coated with an enteric coating.

Bacteria responsive delivery is based on enzymatic activity of bacteria in the lower gastrointestinal tract, especially in the colon, where the bacterial count is approximately 10 million times higher than that in the proximal gastrointestinal tract. The drug to be delivered to the colon is formulated in a compound or matrix that is degraded by enzymes produced and secreted from colonic bacteria. In one embodiment, the solid pharmaceutical product according to the invention is coated with a natural occurring polysaccharide, preferably amylose. In the glassy state, amylose has good film-forming properties and is resistant to degradation by pancreatic enzymes in the small intestines. In combination with water-insoluble polymers which reduce swelling and release of active ingredient of the hydrophilic amylose, e.g. ethylcellulose, a film coating can easily applied to the solid pharmaceutical product formulated as tables or pellets or as a liquid or pellets or granules and filled in capsules.

For the controlled release of active ingredient of the oral solid pharmaceutical product of the invention, pH dependent systems are preferred. The pH of the small intestine increases aborally, and pH sensitive pharmaceutically acceptable excipients and coatings with a dissolution threshold in the range of pH 6.5 to pH 7.5 (distal small intestines, i.e. terminal ileum) are suitable for pH-controlled release of drugs that are to be delivered to the lower intestinal tract, e.g. to the colon. The pH in the terminal ileum is about 1-2 pH units higher than that in the caecum and pH sensitive pharmaceutically acceptable excipients and coatings begin to destabilize and degrade in the region of the terminal ileum/caecum. In a preferred embodiment, the oral solid pharmaceutical product according to the invention is an oral solid pharmaceutical product which provides for the pH controlled release of the active ingredient a) in the range of pH 6.5 to pH 7.5. To achieve this, the solid pharmaceutical product is preferably coated with one or more enteric coatings. Representative examples of materials suitable for use as such coatings include cellulose acetate, hydroxypropyl methylcellulose, copolymers of methacryclic acid and methacrylic esters and polyvinylacetophthalate. Other suitable coatings include cellulose acetate phthalate (CAP), ethylcellulose, dibutyl phthalate and diethyl phthalate. In a preferred embodiment, the enteric coating is an enteric coating comprised of anionic polymers of methacrylic acid and methacrylate (Eudragit®). The Eudragit® grades of polymer which are capable of sustained release are also particularly suitable for use as coating materials. These are based on copolymers of acrylate and methacrylates with quaternary ammonium groups as functional groups as well as ethylacrylate methylmethacrylate copolymers with a neutral ester group. Such polymers are insoluble and permeable and their release profiles can be altered by varying mixing ratios and/or coating thickness. Suitable Eudragit® polymers include the Eudragit® S- and L-types. In a more preferred embodiment, the solid pharmaceutical product is coated with a first and second enteric coating, wherein for said first enteric coating materials selected from cellulose acetate, hydroxypropyl methylcellulose, polyvinylacetophthalate, cellulose acetate phthalate (CAP), ethylcellulose, dibutyl phthalate and diethyl phthalate are used and wherein said second coating is comprised of anionic polymers of methacrylic acid and methacrylate.

As mentioned earlier, it is desired to achieve a high and substantially homogeneous (i.e. uniform) concentration of active ingredient in the lower part of the gastrointestinal system. By regulating the time and place of release of the active ingredient in the colon, and by choosing a suitable triglyceride/emulsifier combination, the desired uniform coverage may be achieved.

Suitable for use in this regard are dosage forms or dosage regimes which comprise a plurality of individual doses, e.g. the pharmaceutical product of the invention in the form of tablets, capsules or a mixture of pellets which are capable of releasing the active ingredient at different rates and/or at different time intervals following administration. The individual doses may be contained within a single dosage form, for example a plurality of nanoparticles, microparticles, pellets, tiny pills, granules or mini-tablets may be provided within a single tablet or capsule in which the individual particles, pellets, pills, granules or mini-tablets are capable of providing different release profiles for the active ingredient. These are generally referred to as “multi-particulate systems”. Alternatively, the dosage may comprise one or more, preferably several, single dose forms, e.g. one or more tablets or capsules intended for separate or simultaneous administration in which the individual single dose forms differ in their release profiles. When examining a patient, it may be envisaged that two or more different doses (e.g. capsules or tablets) containing the active ingredient will be administered which have different release profiles. For example, when using three different capsules it is possible to target the beginning, middle and end of the colon. Due to the peristaltic movement of the colon, the different doses will travel further down the colon thereby assuring a better and more uniform distribution of the active ingredient. In the case where the dosing regime comprises more than one single unit dose, the different unit doses can be administered at the same time or at different time intervals.

The desired release profile may be a delayed release profile and such different release profiles—whether from individual particulates, e.g. pellets, within a single dosage form or from a plurality of single dose forms—may be achieved by any of the means previously described, for example by altering the nature, composition and/or concentration of the triglycerides, emulsifiers and optional pharmaceutically acceptable excipients or by providing a suitable coating. Where a coating is used, the nature of the coating material, its thickness and/or the concentration of the components within the coating may be varied as required to obtain the desired delayed release profile. Where the same coating material is used to coat a plurality of pellets, tablets or capsules, variation in the release profile may be achieved by progressively increasing the concentration of the coating agent used to coat the individual doses resulting in a variation of the thickness of the coating and thus a variation in the release profile. When coated pellets or granules are filled into a capsule or compressed together to form a tablet, the formulation is considered a multi-particulate dosage form. In these, the tablets or capsules containing coated pellets or granules can be further coated, e.g. with a suitable enteric coating, which may be the same or different to that used for coating of the pellets and granules.

Alternatively, a combination of formulations with a rapid and slow release may be used to provide the desired release profile. A suitable dosage regime may, for example, comprise administration of a plurality of capsules or tablets containing different release agents.

The oral dose formulations herein described may, for example, be provided in a pack which comprises a plurality of individual doses having different release profiles. For ease of use, the individual doses (e.g. capsules) may be color coded with different colors. Such packs also form part of the invention.

An advantage of the solid pharmaceutical products of the invention is that they are stable. In particular the active ingredients present within the pharmaceutical products of the invention are not prone to degradation and/or decomposition. As a result, the pharmaceutical products can be stored, e.g. at room temperature and humidity, for at least 6 months, more preferably at least 12 months, still more preferably at least 24 months or more, e.g. up to 36 months.

The solid pharmaceutical products of the present invention are administered orally or by insertion into the rectum. The preferred route of administration will depend on a number of factors including the severity and nature of the cancer, pre-cancerous lesion or non-cancerous condition to be diagnosed, the location of thereof and the nature of the active ingredient.

The photodynamic diagnosis of cancer, pre-cancerous and non-cancerous conditions in the lower part of the gastrointestinal system is usually carried out by endoscopic examination of the lower gastrointestinal tract, i.e. the colon and the distal part of the small bowel with a camera on a flexible tube passed through the anus of a human or non-human animal subject undergoing the endoscopic examination. Apart from diagnosis, it also provides the opportunity for biopsy or removal of suspected lesions or polyps.

The colon must be free of solid matter for the PDD to be performed properly. For one to three days, the subject scheduled for a PDD, i.e. the non-human animal or human, i.e. patient, may be required to follow a low fiber or clear-fluid only diet. The day before the PDD, the bowels need to be cleaned out, a procedure which is commonly called a bowel preparation or bowel prep. Various bowel prep agents are available in solution or in tablet from. Bowel prep tablets contain compounds like bisacodyl and bowel prep solutions contain compounds like sodium phosphate or polyethylene glycol and electrolytes. In a standard regime for a colonoscopy, the amount of bowel prep solution to be ingested is about 4 liters.

On the day of the PDD and preferably 4-12 hours before the endoscopic examination, the oral solid pharmaceutical product according to the invention is ingested according to the prescribed dosage regime, e.g. in single dose of one unit or a single dose of several units or in multiple doses. The patients may be allowed to drink fluid. If the solid pharmaceutical product is a suppository, the suppository is placed at the site of examination. In the case of an examination of the whole lower gastrointestinal tract, the suppository is placed at the distal colon, e.g. caecum.

The time period between administration and endoscopic examination including photoactivation, i.e. exposure of the site of examination to light, will depend on the nature of the pharmaceutical product, its form and the nature of the active ingredient. Generally, it is necessary that the active ingredient within said pharmaceutical product is converted into a photosensitiser and achieves an effective tissue concentration at the site of the examination prior to photo activation.

In a preferred embodiment and to promote that the active ingredient in the pharmaceutical product is distributed to the whole lower gastrointestinal tract the patient is given a “booster” of fluid, preferably a bowel prep solution. The amount of booster is usually between 250 and 750 ml, preferably about 500 ml and the booster is ingested at about 15 min to 90 min, preferably at about 30 to 60 min after the ingestion of the pharmaceutical product according to the invention. In another embodiment, a second booster of fluid, preferably a bowel prep solution may be given to the patient at about 120 min to 150 min after the ingestion of the pharmaceutical product. The uniform distribution of the active ingredient can further be promoted by the patient moving or rolling from one side to another, e.g. lying 10 min on the right side, rolling to the back and lying 10 min on the back, rolling to the left side and lying 10 min on the left side.

During the endoscopic examination, the area to be examined is exposed to light which is suitable for photoactivation, i.e. to achieve the desired photodynamic effect. Areas to be examined are exposed to blue light typically ranging from 380-450 nm. The irradiation will in general be applied at a dose level of 10 to 100 Joules/cm² with an intensity of 20-200 mW/cm² when a laser is used or a dose of 10-100 J/cm² with an intensity of 50-150 mW/cm² when a lamp is applied. The emitted fluorescence (635 nm) is then used to selectively detect affected cancerous tissue or pre-cancerous lesions or other non-cancerous conditions like inflammations. Suitable endoscopes, i.e. colonoscopes are state of the art colonoscopes which are adapted to allow emission of such blue light in addition to white light, e.g. by being equipped with an internal filter assembly which passes primarily blue light. A foot pedal allows convenient switching between white and blue light. The light source may be a laser or a lamp. To visualize fluorescence, the colonoscopes may be equipped with an integrated filter which blocks most of the reflected blue light. A camera like a modified color charge-coupled device (CCD) camera may be used to capture images of the lower gastrointestinal tract and a standard color monitor may be used to display images of the lower gastrointestinal tract. Irradiation is preferably performed for 5 to 30 minutes. A single irradiation may be used or alternatively a light split dose in which the light dose is delivered in a number of fractions, e.g. a few minutes to a few hours between irradiations, may be used. Multiple irradiations may also be applied. The area of examination may further be inspected by use of white light, e.g. before, during or after irradiation with blue light. Polyps, cancerous tissue or pre-cancerous lesions identified due to its fluorescence may be removed during irradiation or in white light.

In a second aspect, the invention provides the use of
a) an active ingredient selected from 5-ALA, a precursor of 5-ALA or a derivative of 5-ALA and pharmaceutically acceptable salts thereof;
b) one or more triglycerides; and
c) one or more emulsifiers

in the manufacture of a solid composition or a solid pharmaceutical product for use in the photodynamic diagnosis of cancer, pre-cancerous and non-cancerous conditions in the lower gastrointestinal tract.

In a preferred embodiment, the invention provides the use of
a) an active ingredient selected from 5-ALA, a precursor of 5-ALA or a derivative of 5-ALA and pharmaceutically acceptable salts thereof;
b) one or more triglycerides; and
c) one or more emulsifiers

in the manufacture of a solid composition or a solid pharmaceutical product for use in the photodynamic diagnosis of cancer and pre-cancerous conditions in the lower gastrointestinal tract, preferably in the colon and rectum.

In a still further aspect the invention provides a method of photodynamic diagnosis of cancer, pre-cancerous and non-cancerous conditions in the lower gastrointestinal tract said method comprising the steps of:
(a) administering to a subject, e.g. a human or non human animal, a solid pharmaceutical product or solid composition as herein defined;
(b) waiting for a time period necessary for the active ingredient within said pharmaceutical product to be converted into a photosensitiser and achieve an effective tissue concentration at the desired site in the lower gastrointestinal tract;
(c) photoactivating the photosensitiser; and
(d) detecting fluorescence from said photosensitiser indicating cancer pre-cancerous and non-cancerous conditions.

The solid pharmaceutical products are novel and hence in a further aspect, the invention provides a solid composition comprising
a) an active ingredient selected from 5-ALA, a precursor of 5-ALA or a derivative of 5-ALA and pharmaceutically acceptable salts thereof;
b) one or more triglycerides; and
c) one or more emulsifiers

The preferred embodiments of the solid compositions are identical with the preferred embodiments of the solid pharmaceutical products for use in the photodynamic diagnosis of cancer, pre-cancerous and non-cancerous conditions in the lower gastrointestinal tract described earlier, i.e. the preferred compounds a), b) and c) and preferred combinations thereof, also described earlier.

In yet another preferred embodiment, the solid composition consists of
a) an active ingredient selected from 5-ALA, a precursor of 5-ALA or a derivative of 5-ALA and pharmaceutically acceptable salts thereof;
b) one or more triglycerides; and
c) one or more emulsifiers

In a preferred embodiment, said solid composition is in the form of a suppository.

Preferably, the solid compositions according the invention are those wherein the active ingredient is a derivative of 5-ALA, preferably a 5-ALA ester or a pharmaceutically acceptable salt thereof, the one or more triglycerides is a solid triglyceride selected from cocoa butter, tallow, hard fat, hydrogenated coco-glycerides, hydrogenated palm oil, tristearin, tripalmitin and trimyristin or a liquid triglyceride selected from triglycerides of glycerol and 3 identical or different C₂⁻C₂₂ fatty acids, more preferably 3 identical or different C₄-C₁₈ fatty acids, even more preferably 3 identical or different C₆-C₁₈ fatty acids and most preferably 3 identical or different C₆-C₁₂ fatty acids, particularly tricaprylin, tricaproin, triheptanoin, caprylic/capric triglyceride, caprylic/capric/linoleic triglyceride and caprylic/capric/succinic triglyceride and the one or more emulsifiers is a non-ionic emulsifier obtained from the reaction of polyethylene glycol and a natural or hydrogenated oil.

The preferred solid compositions are water-free.

In one preferred embodiment, the solid compositions do comprise a liquid, semi-solid or solid mixture of compounds a)-c) filled in solid capsule, preferably in a capsule which is coated with one or more enteric coatings and more preferably which is coated with one or more enteric coatings that provide for the pH controlled release of the active ingredient a) in the range of pH 6.5 to pH 7.5.

In yet another aspect the invention provides a capsule comprising a liquid, semi-solid or solid mixture of compounds a)-c). Preferably, said capsule is coated with one or more enteric coatings and more preferably which is coated with one or more enteric coatings that provide for the pH controlled release of the active ingredient a) in the range of pH 6.5 to pH 7.5. In a preferred embodiment, the invention provides a capsule comprising a liquid, semi-solid or solid mixture consisting of compounds a)-c). Preferably, said capsule is coated with one or more enteric coatings and more preferably which is coated with one or more enteric coatings that provide for the pH controlled release of the active ingredient a) in the range of pH 6.5 to pH 7.5.

In another aspect the invention provides solid compositions or capsules as described above for use as a medicament. In a preferred embodiment, the invention provides solid compositions as described above for use as a medicament.

In yet another aspect the invention provides solid compositions, capsules or suppositories as described above for use in the photodynamic diagnosis of cancer, pre-cancerous and non-cancerous conditions in the lower part of the gastrointestinal system. In a preferred embodiment, the invention provides solid compositions as described above for use in the photodynamic diagnosis of cancer, pre-cancerous and non-cancerous conditions in the lower part of the gastrointestinal system.

### DESCRIPTION OF THE FIGURES

FIG. 1a. shows a 2 hour post-activation gamma scintigraphy image of the gastrointestinal tract of six subjects who were administered with an Enterion™ capsule containing ¹¹In-radiolabelled aqueous solution. The capsule was activated to release its content in terminal ileum/caecum.

FIG. 1b. shows a 6 hour post-activation gamma scintigraphy image of the gastrointestinal tract of the six subjects described above.

FIG. 1c. shows a 12 hour post-activation gamma scintigraphy image of the gastrointestinal tract of the six subjects described above.

FIG. 2a. shows a 2 hour post-activation gamma scintigraphy image of the gastrointestinal tract of four subjects who were administered with an Enterion™ capsule containing a “In-radiolabelled composition consisting of 100 mg HAL HCl in 200 mg Miglyol® 812 and 100 mg Gelucire® 44/14. The capsule was activated to release its content in terminal ileum/caecum.

FIG. 2b. shows a 6 hour post-activation gamma scintigraphy image of the gastrointestinal tract of the four subjects described above.

FIG. 2c. shows a 12 hour post-activation gamma scintigraphy image of the gastrointestinal tract of the four subjects described above.

FIG. 3a. shows a 2 hour post-release gamma scintigraphy image of the gastrointestinal tract of six subjects who were administered with an entero-coated capsule containing a “In-radiolabelled composition consisting of 100 mg HAL HCl in 200 mg Miglyol® 812 and 100 mg Gelucire® 44/14. The capsule disintegrated at pH≧6.5.

FIG. 3b. shows a 6 hour post-activation gamma scintigraphy image of the gastrointestinal tract of the six subjects described above.

FIG. 3c. shows a 12 hour post-activation gamma scintigraphy image of the gastrointestinal tract of the four subjects described above.

The invention is illustrated by the following examples:

### EXAMPLE 1

### Solid Composition According to the Invention

Coated capsules comprising n-hexyl aminolevulinic acid ester (HAL) hydrochloride (HAL HCl) (Photocure ASA, Norway) were prepared by mixing the compounds listed under “capsule composition” at temperatures above their melting points. The mixtures were poured into white HPMC capsules and banded with a mixture of HPMC (3.1 mg), gellan gum (0.015 mg), and tri-sodium citrate (0.05 mg) in water. The capsules were coated with a moisture resistant coating (6.3 mg/cm²), Opadry AMB) followed by an enteric coating (8 mg/cm² with a mixture of 80% Eudragit® L 30 D-55 and 20% Eudragit® FS 30 D, both dispersed in water) to achieve a pH-sensitive film which disintegrates at a pH of 6.5 and above.

| Capsule | | | | | | | | |
|---|---|---|---|---|---|---|---|---|
| composi- | Capsule | | | | | | | |
| tion (mg) | A | B | C | D | E | F | G | H |
| HAL HCl | 100 | 100 | 100 | 100 | 100 | 100 | 100 | 100 |
| Miglyol ® 812 | 300 | 292 | 200 | 260 | 200 | 260 | 200 | 100 |
| Sodium | — | 8 | 100 | — | — | — | — | — |
| Docusate | | | | | | | | |
| Pluronic ® | — | — | — | 40 | 100 | — | — | — |
| L44 | | | | | | | | |
| Gelucire ® | — | — | — | — | — | 40 | 100 | 200 |
| 44/14 | | | | | | | | |

All capsules contained Miglyol 812 as a triglyceride, capsule A is a solid pharmaceutical product outside the scope of the invention, as it does not contain an emulsifier. Capsules B and C contain can anionic emulsifier, capsules D-H contain a non-ionic emulsifier.

### EXAMPLE 2

### Dissolution of HAL

Capsules A-H prepared according to Example 1 were used in an in vitro dissolution study. To mimic the conditions in the human stomach, the capsules were first immersed in 500 ml of a dissolution medium (1) of 0.1 M HCl with a temperature of 37° C. for 1 h. Then the capsules were taken out of this medium and immersed in 500 ml of a dissolution medium (2) of an aqueous phosphate buffer with a pH of 6.5 and a temperature of 37° C. to mimic the conditions in the human terminal ileum, i.e. aqueous environment and pH. For both immersions, a “USP 711” complying dissolution apparatus equipped with paddles and a sinker was used. The capsule was placed in the sinker and immersed in the dissolution medium. A rotation rate of 75 rpm was chosen. 2 ml samples of the dissolution medium were taken manually at 5, 15, 30, 60, 120 and 180 min. The samples were filtered (40 μm HDPE filters) and the HAL content was determined by HPLC. The HAL content of the sample was calculated by comparison with a standard curve. The release of HAL of six capsules A-H was analyzed and the tables below reflect the mean release.

Results:

No HAL release was observed in dissolution medium (1).

Release of HAL was observed in dissolution medium (2) as follows:

| | Release of HAL (% of nominal dose) | | | | | | | |
|---|---|---|---|---|---|---|---|---|
| Time (min) | A | B | C | D | E | F | G | H |
| 5 | 0.0 | 0.3 | 0.3 | 0.4 | 5.0 | 0.0 | 0.0 | 0.0 |
| 15 | 0.0 | 5.7 | 14.5 | 1.7 | 86.4 | 50.2 | 82.4 | 34.8 |
| 30 | 4.8 | 14.6 | 30.5 | 16.4 | 98.5 | 96.4 | 103.6 | 98.3 |
| 60 | 38.6 | 35.0 | 41.9 | 53.7 | 99.2 | 100.2 | 104.8 | 99.2 |
| 120 | 61.4 | 45.7 | 49.8 | 66.4 | 99.3 | 100.3 | 100.2 | 98.8 |
| 180 | 59.2 | 47.8 | 54.4 | 66.0 | 98.9 | 100.3 | 99.3 | 99.1 |

The content of all capsules was instantly released into the dissolution medium (2).

After release of the content of capsule A into the dissolution medium, an inconsistent release of HAL from the composition with high levels of variability was observed. The released composition floated on the surface of the dissolution medium.

The compositions of capsules B and C did not show an improved HAL release profile compared to the composition of capsule A. As capsule A, they demonstrated a release profile consisting of steady increase of HAL release over the first 120 min followed by a plateau in concentration of HAL for the remaining 60 minutes. After 180 min, oily globules were present on the surface of the dissolution medium. It is apparent that the presence of the anionic emulsifier sodium docusate in a concentration of 2% and 25% did not promote the spreading/dissolution of HAL in the aqueous dissolution medium.

The composition of capsule D demonstrated a slightly larger release of HAL from the formulation. However, after 180 min, oily globules were present on the surface of the dissolution medium. It is apparent that the presence of the non-ionic emulsifier Pluronic® L44 in a concentration of 10% did—to some degree—promote the spreading/dissolution of HAL in the aqueous dissolution medium.

The composition of capsule E exhibited a markedly different release profile of HAL, with a full release after 30 min and with about 80% release after 15 min. No oily globules were observed on the surface of the dissolution medium. It is apparent that that the presence of the non-ionic emulsifier Pluronic® L44 in a concentration of 25% did significantly promote the spreading/dissolution of HAL in the aqueous dissolution medium.

The compositions of capsules F to H all demonstrated full release of HAL within 30 min. Visual observations suggested that an emulsion was formed upon release, which subsequently coalesced into an oily layer upon the surface of the dissolution medium. The test results confirmed that this effect was sufficient to allow the dissolution of HAL in the dissolution medium. It is apparent that that the presence of the non-ionic emulsifier Gelucire® 44/14 in a concentration of 10%, 25% and 50% did significantly promote the spreading/dissolution of HAL in the aqueous dissolution medium. The results further indicate that no increase in HAL dissolution is achieved by a concentration of Gelucire® 44/14 above 25%. Also, an increased amount of HAL was released from the composition within 15 in the presence of 25% Gelucire® 44/14 then in the presence of 10% Gelucire® 44/14 (82.4% vs. 50.2%). This indicates that the amount of emulsifier does not only impact the dissolution of HAL in an aqueous phase but also has an impact on the speed of release of HAL from the composition.

### EXAMPLE 3

### Release of HAL HCl In Vivo

A gamma scintigraphy study was carried out in healthy male volunteers to assess gastrointestinal transit of an entero-coated capsule, i.e. a solid pharmaceutical product according to the invention, to determine the site of release of the composition from said an entero-coated capsule and the distribution of HAL HCl in the empty, i.e. cleansed, colon.

Gamma scintigraphic imaging permits the assessment of the physical integrity of the solid pharmaceutical product according to the invention, as it transits through the gastrointestinal tract. Detailed information on the timing and anatomical location of disintegration of the product can be obtained. An Enterion™ site-specific delivery capsule was used for the gamma scintigraphy study, which allows the targeted regional drug delivery to the gastrointestinal tract by. The capsule is 35 mm in length and 10-12 mm in diameter and is capable of delivering solutions, suspensions or powders to specific sites. The location of the capsule in the gastrointestinal tract is determined using gamma scintigraphy. The capsule contains a drug chamber which is loaded with the formulation under assessment. Capsules are activated and the formulation released, using a low strength electromagnetic field generated by an activation unit. Capsule activation is confirmed by means of a signal that is emitted from the capsule when activation occurs and is relayed back to the activation unit. Indium-111 is used as a marker in the capsule to allow tracking of the capsule throughout the gastrointestinal tract. The water-soluble radioactive marker, technetium-diethylene triaminepentaacetic acid (^{99m}Tc-DTPA), is mixed with the water that is taken with the capsule to provide visual (scintigraphic) confirmation of the subject's gastrointestinal anatomy.

All subjects underwent bowel cleansing: MoviPrep was administered on the evening before dosing to cleanse the bowel prior to dosing the following morning.

Control Group:

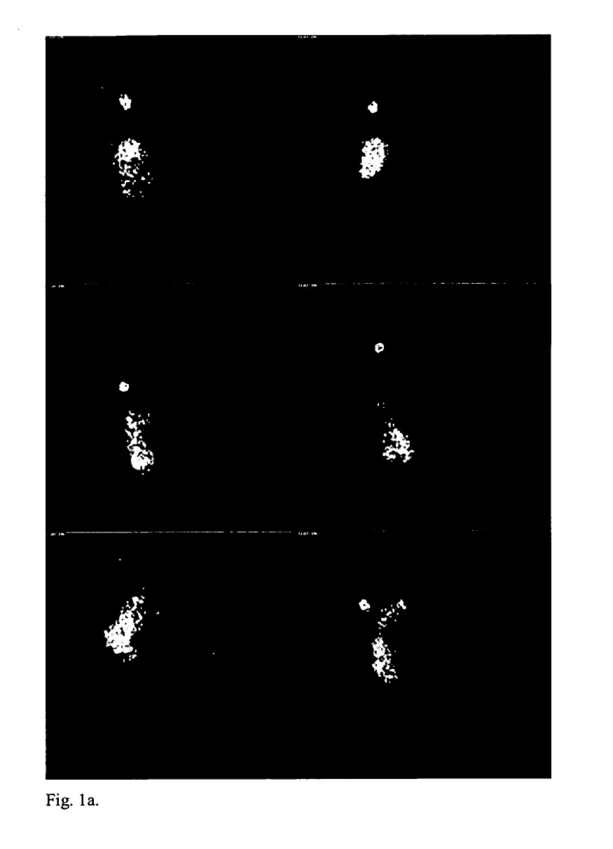

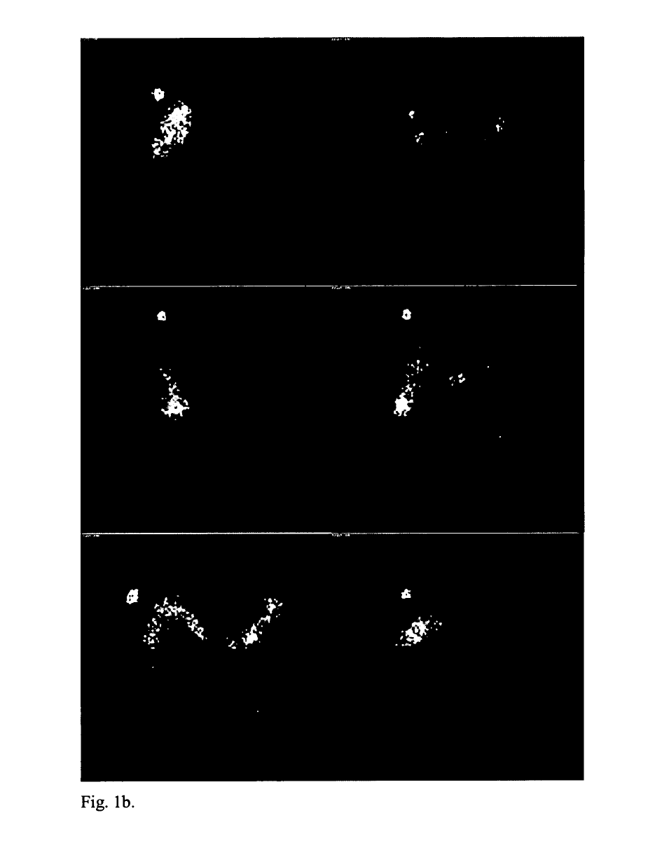

A group of 6 subjects received treatment A, an aqueous formulation radiolabelled with not more than 1 MBq ¹¹¹In-DTPA (formulation A), delivered via the Enterion™ capsule to the terminal ileum/caecum, where its content was released. The Enterion™ capsule was delivered with a total of 500 mL water radiolabelled with not more than 4 MBq ^{99m}Tc-DTPA. The ^{99m}Tc-labelled water was ingested in 2 aliquots of 250 mL; the first aliquot on administration of the capsule and the second after gastric emptying of the capsule. Gamma scintigraphy was carried out and the extent of spread of the formulation A throughout the colon was determined. At 1 hour post-activation, spread was limited and confined to the caecum and ascending colon. Maximal spread, i.e. spread in the whole colon was observed at average of 7 hour post-activation. FIG. 1a-c show the spread at 2, 6 and 12 hours post-activation.

The spread of formulation A was assumed to be the “best-case-situation”, it is the spread of a relatively hydrophilic compound (¹¹¹In-DTPA) formulated in an aqueous solution in an aqueous environment (colon) and thus will resemble the spread of the relatively hydrophilic compound HAL HCl formulated in an aqueous solution in an aqueous environment (colon).

Treatment Group 1:

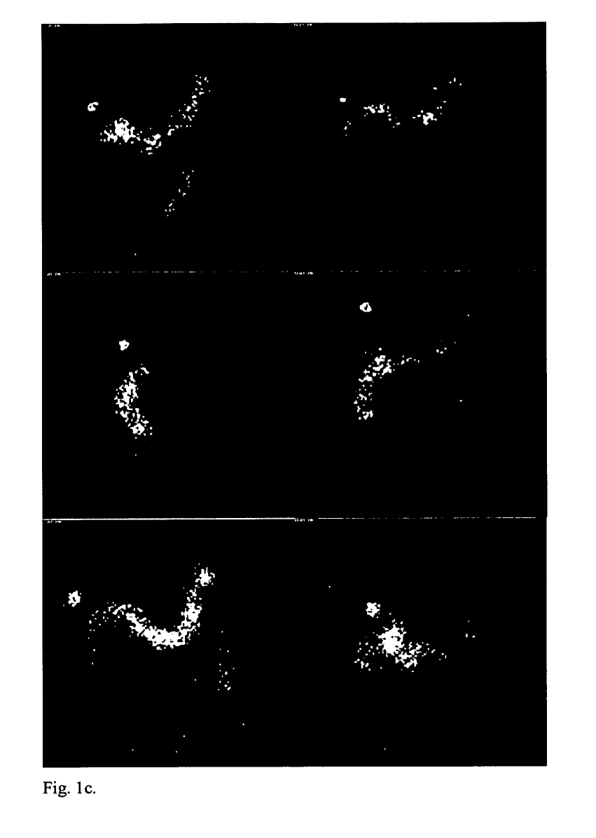

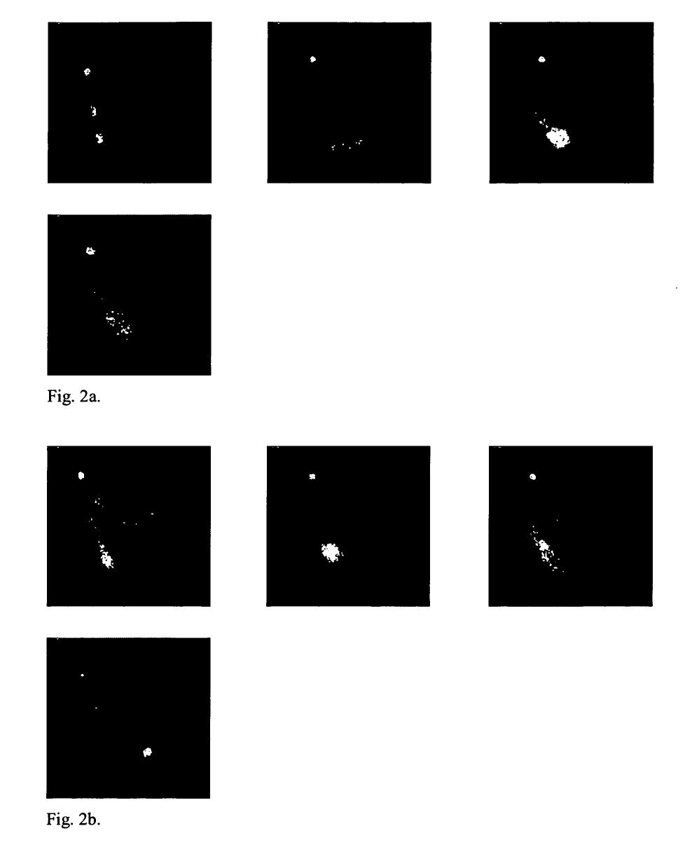

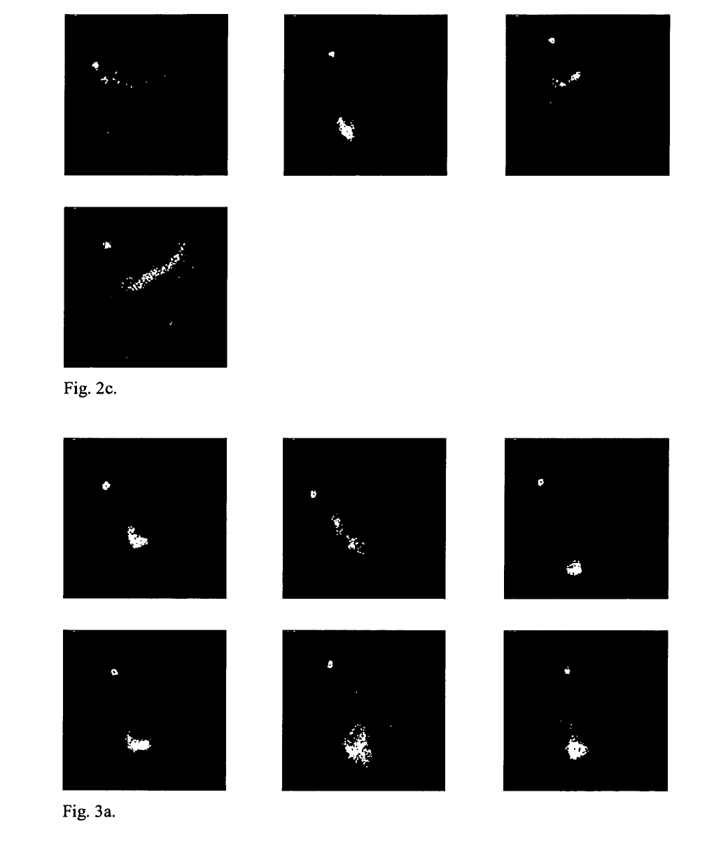

A group of 4 subjects received treatment B, a composition of 100 mg HAL HCl in 200 mg Miglyol® 812 and 100 mg Gelucire® 44/14 radiolabelled with not more than 1 MBq ¹¹¹In-DTPA (composition B), delivered via the Enterion™ capsule to the terminal ileum/caecum, where its content was released. The Enterion™ capsule was delivered with a total of 500 mL water radiolabelled with not more than 4 MBq ^{99m}Tc-DTPA. The ^{99m}Tc-labelled water was ingested in 2 aliquots of 250 mL; the first aliquot on administration of the capsule and the second after gastric emptying of the capsule. Gamma scintigraphy was carried out and the extent of spread of the formulation B throughout the colon was determined. At 1 hour post-activation, colon arrival of the composition had only occurred in 1 subject. Maximal spread, i.e. spread in the whole colon was observed at average of 10 hour post-activation. It was found that the spread of composition B was similar to that of formulation A, i.e. as good as the spread of water, see FIG. 2a-c, which show the spread at 2, 6 and 12 hours post-activation.

Treatment Group 2:

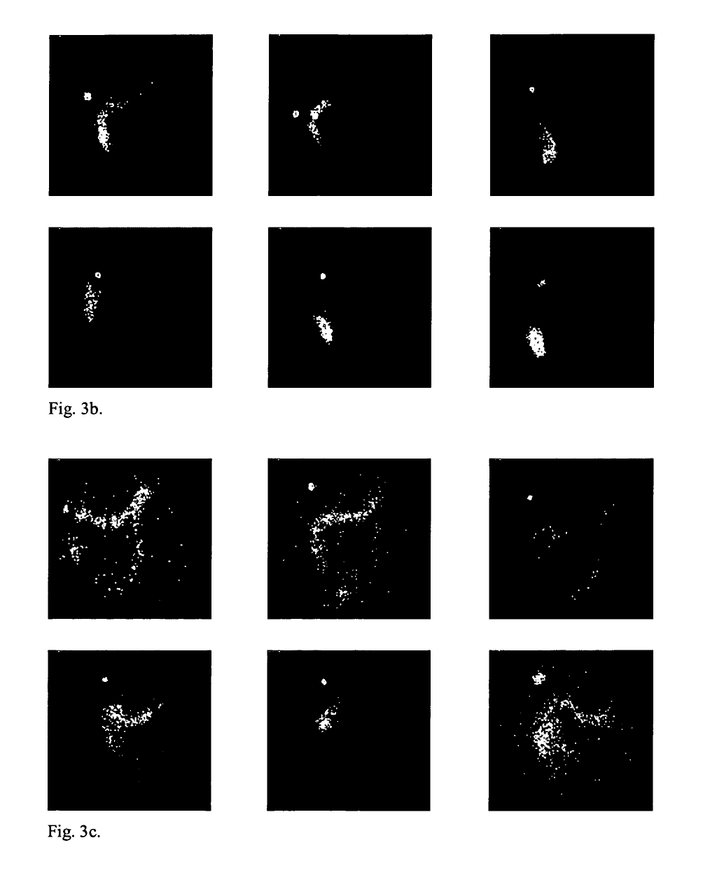

A group of 6 subjects received treatment C, a composition of 100 mg HAL HCl in 200 mg Miglyol® 812 and 100 mg Gelucire® 44/14 radiolabelled with not more than 1 MBq ¹¹¹In-DTPA (composition C), delivered via an entero-coated capsule as described in Example 1. The entero-coated capsule was delivered with a total of 500 mL water radiolabelled with not more than 4 MBq ^{99m}Tc-DTPA. The ^{99m}Tc-labelled water was ingested in 2 aliquots of 250 mL; the first aliquot on administration of the capsule and the second after gastric emptying of the capsule. Gamma scintigraphy was carried out the extent of spread of the formulation C throughout the colon was determined. It was found that the spread of composition C was similar to that of formulation A, i.e. as good as the spread of water, see FIG. 3a-c which show the spread at 2, 6 and 12 hours post-release.

1. Solid pharmaceutical product for use in the photodynamic diagnosis of cancer, pre-cancerous and non-cancerous conditions in the lower gastrointestinal tract comprising
a) an active ingredient selected from 5-ALA, a precursor of 5-ALA or a derivative of 5-ALA and pharmaceutically acceptable salts thereof;
b) one or more triglycerides; and
c) one or more emulsifiers.

2. Solid pharmaceutical product according to claim 1 wherein said active ingredient is a derivative of 5-ALA, preferably a 5-ALA ester or a pharmaceutically acceptable salt thereof.

3. Solid pharmaceutical product according to claim 1 wherein said active ingredient is a compound of formula I or an acceptable salt thereof
R²₂N—CH₂COCH₂—CH₂CO—OR¹ (I)
wherein
R¹ represents a substituted or unsubstituted alkyl group; and
R² each independently represents a hydrogen atom or a group R¹.

4. Solid pharmaceutical product according to claim 1 wherein the one or more triglycerides are triglycerides of glycerol and 3 identical or different C₂-C₂₂ fatty acids, more preferably 3 identical or different C₄-C₁₈ fatty acids, even more preferably 3 identical or different C₆-C₁₈ fatty acids and most preferably 3 identical or different C₆-C₁₂ fatty acids.

5. Solid pharmaceutical product according to claim 1 wherein the one or more triglycerides are solid triglycerides selected from cocoa butter, tallow, hard fat, hydrogenated coco-glycerides, hydrogenated palm oil, tristearin, tripalmitin and trimyristin or liquid triglycerides selected from tricaprylin, tricaproin, triheptanoin, caprylic/capric triglyceride caprylic/capric/linoleic triglyceride.

6. Solid pharmaceutical product according to claim 1 wherein the one or more emulsifier is a non-ionic.

7. Solid pharmaceutical product according to claim 1 wherein the one or more emulsifier is a non-ionic emulsifier obtained from the reaction of polyethylene glycol and a natural or hydrogenated oil.

8. Solid pharmaceutical product according to claim 1 further comprising
d) optionally one or more mucoadhesives
e) optionally one or more pharmaceutically acceptable excipients other than b), c) and d);
f) optionally one or more surface penetration agents; and
g) optionally one or more chelating agents.

9. Solid pharmaceutical product according to claim 1 wherein said product is a suppository or an oral solid pharmaceutical product comprising one or more enteric coatings and which provides for the pH controlled release of the active ingredient a) in the range of pH 6.5 to pH 7.5.

10. Solid pharmaceutical product according to claim 1 wherein said solid pharmaceutical product is water-free.

11. Solid composition comprising
a) an active ingredient selected from 5-ALA, a precursor of 5-ALA or a derivative of 5-ALA and pharmaceutically acceptable salts thereof;
b) one or more triglycerides; and
c) one or more emulsifiers.

12. Solid compositions according to claim 11 wherein the active ingredient is a derivative of 5-ALA, preferably a 5-ALA ester or a pharmaceutically acceptable salt thereof, the one or more triglycerides is a solid triglyceride selected from cocoa butter, tallow, hard fat, hydrogenated coco-glycerides, hydrogenated palm oil, tristearin, tripalmitin and trimyristin or a liquid triglyceride selected from triglycerides of glycerol and 3 identical or different C₂-C₂₂ fatty acids, more preferably 3 identical or different C₄-C₁₈ fatty acids, even more preferably 3 identical or different C₆-C₁₈ fatty acids and most preferably 3 identical or different C₆-C₁₂ fatty acids and the one or more emulsifiers is a non-ionic emulsifier obtained from the reaction of polyethylene glycol and a natural or hydrogenated oil.

13. Solid composition according to claim 11 for use as a medicament.

14. Solid composition according to claims 11 in the photodynamic diagnosis of cancer, pre-cancerous and non-cancerous conditions in the lower gastrointestinal tract.

15. Solid pharmaceutical product according to claim 1 for use in a method of photodynamic diagnosis of cancer, pre-cancerous and non-cancerous conditions in the lower gastrointestinal tract said method comprising the steps of:
(a) administering to a subject, e.g. a human or non human animal, a solid pharmaceutical product of claim 1;
(b) waiting for a time period necessary for the active ingredient within said pharmaceutical product to be converted into a photosensitiser and achieve an effective tissue concentration at the desired site in the lower gastrointestinal tract;
(c) photoactivating the photo sensitiser; and
(d) detecting fluorescence from said photosensitiser indicating cancer precancerous and non-cancerous conditions.
